(19) United States
(12) Patent Application Publication
(10) Pub. No.: US 2008/0015470 A1
(43) Pub. Date: Jan. 17, 2008

(54) Sample tube for receiving body fluids, particularly blood

(76) Inventor: Walter Sarstedt, Numbrecht (DE)

Correspondence Address: K.F. ROSS P.C., 5683 RIVERDALE AVENUE, SUITE 203 BOX 900, BRONX, NY, 10471-0900, US

(21) Appl. No.: 11/805,451

(22) Filed: May 23, 2007

(30) Foreign Application Priority Data
Nov. 23, 2004 (DE) 102004056655.0
Oct. 21, 2005 (DE) PCT/DE2005/001887

(51) Int. Cl. A61B 5/15

(52) U.S. Cl. 600/584

(57) ABSTRACT

A specimen tube for receiving bodily fluids, particularly blood, has a sealing cap having a tip with a pierceable membrane, stopper or similar device, at its end. A guide sleeve can be placed on the tip and carries a single or double needle. The tube can also be provided with an emergency indicator that is configured as an emergency signal colored ring surrounding the tip but leaving free access from outside to the pierceable element at the end of the tip.

The invention relates to a specimen tube for receiving bodily fluids, particularly blood, comprising a sealing cap having a tip with a pierceable membrane provided in the end of the tip, for a guide sleeve that can be placed on the tip and carrying a single cannula or double cannula, which specimen tube can be provided with a colored emergency indicator.

In a blood-drawing device known from DE 30 49 503 C [U.S. Pat. No. 4,449,539], the cap closing the extraction tube on its front end has a cylindrical tip projecting in an axial direction. On its front end, the tip is closed with a pierceable stopper/membrane resting on the front plate of the tip, which plate is provided with a center hole, and being held by a collar that is flanged at the front end. The sealing cap is preferably screwed onto the specimen tube, or alternatively slipped on. The tubular guide sleeve, which on its front end carries in a bracket a double-ended cannula with a two-sided cutting edge, the end projecting from the guide sleeve serving for vein insertion, while its rear end extends so far into the guide sleeve that it pierces the membrane/stopper when applying the guide sleeve on the specimen tube, is disposed axially displaceably and rotatably on the tip. The rear end of the cannula extending into the guide sleeve is enclosed by a bag-like tube (valve rubber) having such a length that the cutting edge of the rear cannula end does not touch its bottom when the tube is extended. For connection of the double cannula to the tip, in the known blood-drawing device the tip is provided with laterally protruding retaining bumps with which axial slots in the guide sleeve can fit. By means of the retaining bump inserted in one of the axial slots distributed around the circumference, a bayonet cap-like twistlock configuration of the double cannula guide sleeve loosely fitting on the tip may be achieved.

In another known embodiment of a sealing cap (also compare EP 0 818.296 B1 [U.S. Pat. No. 5,997,275]) that can be slipped or screwed on the open end of a specimen tube, three retaining bumps offset by 90° in relation to each other are provided for locking of the guide sleeve.

During emergencies, when the collected blood must be quickly analyzed, it is commonly known to provide the corresponding specimen tube with an emergency indicator, so that the emergency situation and the great need for urgency is instantly and immediately obvious to the operating personnel. For this purpose, it has been common for many years to provide such a specimen tube with a colored label or to apply a colored membrane cap to the tip of the sealing cap. The one as well as the other of these measures is associated with the disadvantage that the colored emergency coding of a specimen tube, particularly in the case of specimen tubes that are inserted into a common stand along with countless other specimen tubes not marked as emergencies, is not as evident as the emergency requires. After all, specimen tubes with labels do not stand out in the stand holder, and neither do the membrane caps, which are only slipped onto the retaining bumps of the tip and therefore offer a signal on a small surface. Also, while a specimen tube marked with a colored membrane cap may be accessible for direct adaptation during analysis, the sampling needle penetrating the specimen tube for one must penetrate the cap membrane and secondly the membrane of the sealing cap tip. The force required to overcome a large resistance may result in the destruction of the membrane cap.

Therefore, it is the object of the invention to create a generic specimen tube without the above-mentioned disadvantages, which tube particularly provides reliable recognition of the emergency designation and additionally offers greater reliability during the analysis of the collected bodily fluid samples.

This object is achieved according to the invention in that the emergency indicator is configured as an emergency signal colored ring surrounding the tip covering with free access from above to the end of the tip. Thus, several advantages can be achieved simultaneously. Namely, by means of the signal colored ring substantially surrounding the tip a maximum signal surface is provided that almost corresponds to the total circumferential surface of the tip. For this purpose, the signal colored ring has a tubular end section that is enlarged in its diameter so that it may be slipped over the retaining bump(s) of the tip, while a head section of the signal colored ring fits tightly on the outer circumference of the tip.

Advantageously, the preferably fluorescent signal colored ring ends directly in front of the normally tapered tip end. The exposed cone end of the tip end helps maintain the specimen tube's ability to be centered, which is required for analysis of the collected sample. Finally, direct adaptation of the specimen tube marked with a signal colored ring according to the invention is possible without having to penetrate two stoppers or membranes.

Additional details and characteristics of the invention will be apparent from the claim and the following description of one embodiment of the invention illustrated in the figures, wherein:

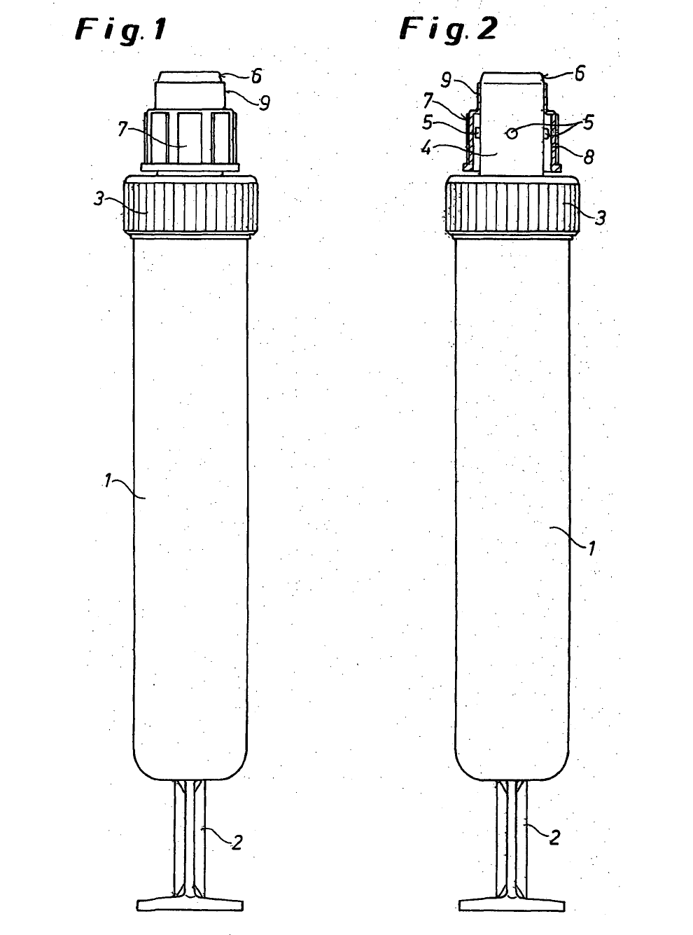

FIG. 1 is a detail of a blood-drawing device in a general view of a specimen tube with an emergency signal colored ring slipped on the tip of the sealing cap, and

FIG. 2 is an illustration as above, however with the signal colored ring in a longitudinal sectional view.

In FIGS. 1 and 2, a cylindrical specimen tube 1 of a blood-drawing device is shown in a larger scale, in which a plunger rod 2 with a plunger is disposed displaceably in an airtight way. In this embodiment, the specimen tube 1 is sealed on its front end by a screw-on sealing cap 3. From it a tip 4 extends axially having retaining bumps 5 (see FIG. 2), serving for securing an unillustrated guide sleeve carrying a single or double needle. A membrane or stopper (not shown) is provided in the conical tip end 6.

For marking the specimen tube 1 as an emergency, a signal colored ring 7, for example in a fluorescent color, is slipped on the tip 4 as a indicator. It is also manufactured in one piece from plastic by means of injection molding and comprises a tubular end section 8, which is of larger diameter than the tip, and a head section 9 that tapers upward. When the signal colored ring 7 is slipped onto the tip 4, the tubular end section 8 slides over the retaining bumps 5 and extends down to the threaded part of the cap 3, while the head section 9 extends down to the end of the cone and tightly fits against the outer circumference of the tip 4. Thus, the signal colored ring 7 surrounds the entire surface of the tip and therefore offers the largest possible signal surface, which is accordingly easy to recognize even if such a specimen tube 1 is placed in a stand together with specimen tubes not marked for emergency.

The free cone end of the tip end 6 guarantees good centering ability of the specimen tube 1 marked as an emergency. In addition, the membrane provided in the tip end 6 is freely accessible from above despite the large signal designation, thus making the specimen tube 1 marked as an emergency immediately accessible for safe direct adaptation in an analytical apparatus.

1. A specimen tube (1) for receiving bodily fluids, particularly blood, comprising a sealing cap (3) having a tip (4) with a pierceable membrane, stopper or similar device, which is provided in the end of the tip (6), for a guide sleeve that can be placed on the tip and bears a cannula or double cannula, which tube can be provided with an emergency indicator, characterized in that the emergency indicator is configured as an emergency signal colored-ring (7) surrounding the tip covering with free access from above to the end of the tip (6).

2. In combination:
a blood-specimen tube comprising
a tubular body-extending along an axis and having an axially extending small-diameter tip, and
an axially pierceable element at the tip;
an emergency indicator comprising
a sleeve of a predetermined signal color and dimensioned to fit complementary around the tip while leaving the pierceable element exposed.

3. The combination defined in claim 2 wherein the tip is provided with radially outwardly projecting latch bumps, the sleeve being stepped and having a large-diameter inner portion fitting over the bumps and a small-diameter portion closely surrounding the tip outward of the bumps.

4. The combination defined in claim 2, further comprising
an outer portion formed with the tip and forming a nut threaded to an end of the body.

5. The combination defined in claim 2, further comprising
a guide sleeve fittable complementarily over the tip, and
a needle fitted to the sleeve and pierceable through the element when the sleeve is fitted to the tip.

6. The combination defined in claim 2 wherein the pierceable element is a membrane or plug.
